(19) United States
(12) Patent Application Publication
(10) Pub. No.: US 2024/0358499 A1
(43) Pub. Date: Oct. 31, 2024

(54) PROSTHETIC HEART VALVES

(71) Applicant: Medtronic, Inc., Minneapolis, MN (US)

(72) Inventor: Karl L. OLNEY, Santa Ana, CA (US)

(73) Assignee: Medtronic, Inc., Minneapolis, MN (US)

(21) Appl. No.: 18/533,327

(22) Filed: Dec. 8, 2023

Related U.S. Application Data
(60) Provisional application No. 63/462,513, filed on Apr. 27, 2023.

(51) Int. Cl. A61F 2/24

(52) U.S. Cl. CPC A61F 2/2418; A61F 2230/0069; A61F 2250/0058

(57) ABSTRACT

Prosthetic heart valves each includes a link extending along an axial direction and an axial frame. The axial frame includes a plurality of struts comprising a plurality of inner struts pivotally attached to a plurality of outer struts at a plurality of pivot nodes. A first pivot node of the plurality of pivot can be attached to the link and a second pivot node of the plurality of pivot nodes can move relative to the link in the axial direction. Methods of radially expanding a prosthetic heart valve can comprise radially expanding the radially expandable frame from a radially retracted orientation to a radially expanded orientation while the second pivot node axially translates relative to the link.

### CROSS-REFERENCE TO RELATED APPLICATIONS

This application claims the benefit of U.S. Provisional Patent Application Ser. No. 63/462,513, filed Apr. 27, 2023, the entire content of which is incorporated herein by reference.

### FIELD

The present disclosure relates generally to prosthetic heart valves and, more particularly, to prosthetic heart valves that comprise a radially expandable frame comprising a plurality of struts and a link pivotally attached to a first pivot node and a second pivot node of the radially expandable frame.

### BACKGROUND

A human heart includes four heart valves that determine the pathway of blood flow through the heart: the mitral valve, the tricuspid valve, the aortic valve, and the pulmonary valve. The mitral and tricuspid valves are atrioventricular valves, which are positioned between the atria and the ventricles, while the aortic and pulmonary valves are semilunar valves, which are positioned in the arteries leaving the heart. Ideally, native leaflets of a heart valve move apart from each other when the valve is in an open position, and include coaptation edges that meet or “coapt” when the valve is in a closed position. Valves may develop stenosis in which a valve does not open properly, and/or insufficiency or regurgitation in which retrograde blood flow occurs because a valve does not close properly. Stenosis and insufficiency may occur simultaneously in the same valve. The effects of valvular dysfunction vary, with insufficiency or regurgitation typically having relatively severe physiological consequences to the patient.

It is known to provide prosthetic heart valves comprising valve leaflets and a valve skirt supported by a radially expandable frame that can be delivered percutaneously using a catheter-based delivery system to replace a native heart valve. Known annular frames include pivoting struts that mechanically pivot relative to one another at pivot nodes. Sutures may mount the valve skirt and valve leaflets within an interior of the radially expandable frame. The prosthetic heart valves can be reduced in diameter and thereafter contained within a sheath of a delivery catheter, and advanced through the venous or arterial vasculature to the treatment site. Once the prosthetic valve is positioned at the treatment site, for instance within a defective native heart valve, the radially expandable frame may be mechanically expanded to hold the prosthetic valve firmly in place while the valve skirt and valve leaflets are deployed to functionally replace the native heart valve.

Annular frames with pivoting struts may create problems by inadvertently cutting or pinching the valve skirt, valve leaflets, and/or corresponding sutures due to a scissor-action between engaged edges of pivoting strut segments as the radially expandable frame is dilated or collapsed. There is a need to provide prosthetic heart valves that mechanically dilate and contract without damaging the skirts, leaflets and/or sutures mounting the skirts and leaflets relative to the radially expandable frames.

### SUMMARY

The following presents a simplified summary of the disclosure to provide a basic understanding of some aspects described in the detailed description.

In aspects, a prosthetic heart valve comprises a link extending along an axial direction and an axial frame. The axial frame comprises a plurality of struts comprising a plurality of inner struts pivotally attached to a plurality of outer struts at a plurality of pivot nodes. The axial frame further comprises a first pivot node of the plurality of pivot nodes pivotally connecting a first inner strut of the plurality of inner struts to a first outer strut of the plurality of outer struts at the first pivot node. The first pivot node is attached to the link. The axial frame further comprises a second pivot node of the plurality of pivot nodes pivotally connecting a second inner strut of the plurality of inner struts to a second outer strut of the plurality of outer struts at the second pivot node. The second pivot node is slidably connected to the link, wherein the second pivot node is configured to move relative to the link along the axial direction.

In further aspects, a heart valve comprises a link comprising an axial slot extending along an axial direction, and a radially expandable frame. The radially expandable frame comprises a plurality of struts comprising a plurality of inner struts pivotally attached to a plurality of outer struts at a plurality of pivot nodes. The radially expandable frame further comprises a first pivot node of the plurality of pivot nodes comprising a first pivot pin extending through an aperture of a first inner strut of the plurality of inner struts and an aperture of a first outer strut of the plurality of outer struts to pivotally connect the first inner strut to the first outer strut at the first pivot node. The first pivot node is attached to the link. The radially expandable frame further comprises a second pivot node of the plurality of pivot nodes pivotally connecting a second inner strut of the plurality of inner struts to a second outer strut of the plurality of outer struts at the second pivot node. The second pivot node comprises a second pivot pin extending through the axial slot to slidingly connect the second pivot node to the link. The second pivot node is configured to move relative to the link along the axial direction.

In further aspects, a method is provided for radially expanding a prosthetic heart valve comprising a radially expandable frame comprising a plurality of struts comprising a plurality of inner struts pivotally attached to a plurality of outer struts at a plurality of pivot nodes, wherein a first pivot node of the plurality of pivot nodes is connected to a link and a second pivot node of the plurality of pivot nodes is slidably connected to the link. The method comprises pivoting a first inner strut of the plurality of inner struts relative to a first outer strut of the plurality of outer struts at the first pivot node, wherein the first inner strut and the first outer strut pivot in opposite directions about a first pivot axis of the first pivot node. The method further comprises pivoting a second inner strut of the plurality of inner struts relative to a second outer strut of the plurality of outer struts at a second pivot node, wherein the second inner strut and the second outer strut pivot in opposite directions about a second pivot axis of the second pivot node while the second pivot node translates relative to the link in the axial direction while being slidably connected to the link. The radially expandable frame expands from a radially retracted orientation to a radially expanded orientation.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 is a perspective view of a prosthetic heart valve in accordance with aspects of the disclosure;

FIG. 2 is an enlarged view of a first portion of the prosthetic heart valve taken at view 2 of FIG. 1 when the prosthetic heart valve is in a radially expanded orientation;

FIG. 3 is a cross-section of the first portion of the prosthetic heart valve taken along lines 3-3 of FIG. 2 when the prosthetic heart valve is in the radially expanded orientation;

FIG. 4 is an enlarged view of a first pivot node of a column of pivot nodes of the first portion of the prosthetic heart valve taken at view 4 of FIG. 3;

FIG. 5 is an enlarged view of another embodiment of a first pivot node of the column of pivot nodes of the first portion of the prosthetic heart valve taken at views 4 of FIG. 3;

FIG. 6 is an enlarged view of a second pivot node of the column of pivot nodes of the first portion of the prosthetic heart valve taken at view 6 of FIG. 3;

FIG. 7 is an enlarged view of intermediate nodes of the column of pivot nodes of the first portion of the prosthetic heart valve taken at views 7 of FIG. 3;

FIG. 8 is an enlarged view of the first portion of the prosthetic heart valve of FIG. 2 when the prosthetic heart valve is in a radially collapsed orientation;

FIG. 9 is a cross sectional view of the first portion of the prosthetic heart valve in the radially collapsed orientation taken along line 9-9 of FIG. 8;

FIG. 10 is a cross-section of further portion of the prosthetic heart valve taken along line 10-10 of FIG. 1 when the prosthetic heart valve is in a radially expanded orientation;

FIG. 11 is an enlarged view of a second pivot node of a column of pivot nodes of the further portion of the prosthetic heart valve taken at view 11 of FIG. 10;

FIG. 12 is a cross-sectional view of the second portion of the prosthetic heart valve of FIG. 10 when the prosthetic heart valve is in a radially collapsed orientation;

FIG. 13 is an enlarged view, similar to view 2 of FIG. 1, but illustrating a first portion of another embodiment of the prosthetic heart valve in a radially expanded orientation;

FIG. 14 is a cross-section of along line 13-13 of FIG. 13 of the prosthetic heart valve of FIG. 13 in the radially expanded orientation;

FIG. 15 is an enlarged view of an intermediate pivot node of the column of pivot nodes of taken at view 15 of FIG. 14; and

FIG. 16 is an enlarged view of another intermediate pivot node of the column of pivot nodes of taken at view 16 of FIG. 14.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

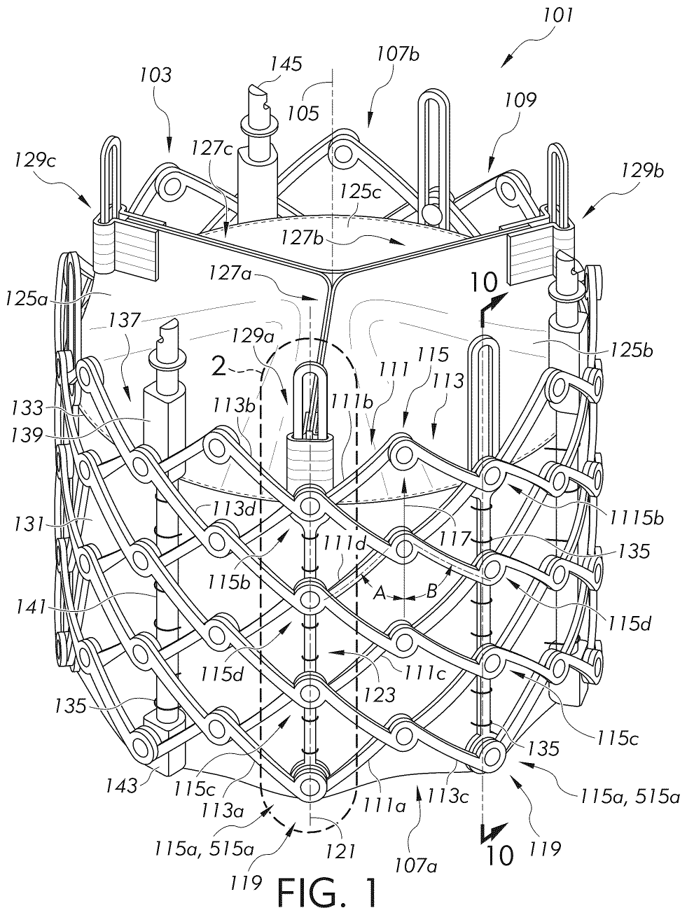

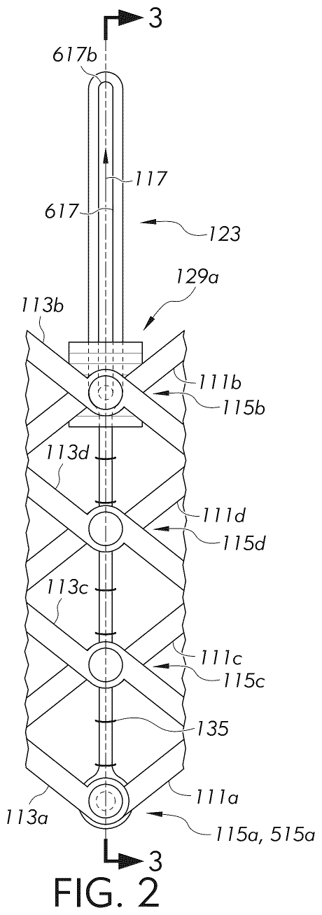

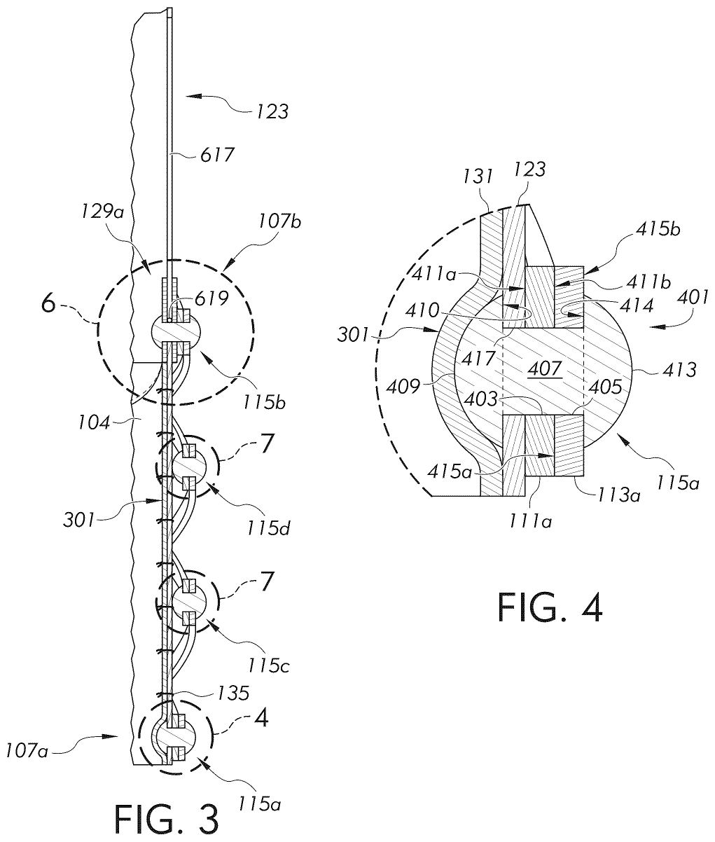

FIG. 1 illustrates a prosthetic heart valve 101 in accordance with aspects of the disclosure. The prosthetic heart valve 101 comprises a radially expandable frame 103 extending along a valve axis 105 between an inflow end 107a and an outflow end 107b of the radially expandable frame 103. Throughout the disclosure, the inflow end and outflow end designate the direction of blood flow through the prosthetic heart valve in use. With reference to FIG. 1, in use, the blood flow enters the inflow end 107a of the prosthetic heart valve 101, passes through an inner area 104 (see FIG. 3) defined by the radially expandable frame 103, and then exits through the outflow end 107b of the prosthetic heart valve 101.

The radially expandable frame 103 comprises a plurality of struts 109 comprising a plurality of inner struts 111 pivotally attached to a plurality of outer struts 113 at the plurality of pivot nodes 115 that are configured to allow the plurality of struts 109 to rotate relative to the pivot nodes 115 such that the radially expandable frame 103 can radially dilate from a collapsed orientation to an expanded orientation shown in FIG. 1. The plurality of pivot nodes 115 can be further configured to allow the radially expandable frame 103 to radially collapse from the illustrated expanded orientation to the collapsed orientation. The plurality of inner struts 111 are positioned radially closer to the valve axis 105 relative to the position of the plurality of outer struts 113 that is positioned radially outward from the plurality of inner struts 111 relative to the valve axis 105. Each inner strut of plurality of inner struts 111 can be circumferentially parallel relative to one another in the expanded orientation with an acute angle “A” relative to an axial direction 117 of the valve axis 105. Each outer strut of the plurality of outer struts 113 can be circumferentially parallel relative to one another in the expanded orientation with an acute angle “B” relative to the axial direction 117 of the valve axis 105. The acute angles “A,” “B” can be substantially identical to one another and mirror images of one another about an axis extending through a center the corresponding pivot node in the axial direction 117 of the valve axis 105. In some embodiments, the struts 109 can each comprise a one-piece unitary strut that extends along a strut axis from the inflow end 107a to the outflow end 107b of the radially expandable frame 103. Each one-piece unitary strut can comprise a plurality of apertures that can be equally spaced along the strut axis, where the apertures define, at least in part, the plurality of pivot nodes 115. The one-piece unitary struts can comprise a wide range of materials such as stainless steel, nickel, cobalt, chromium, nickel titanium alloy or nitinol, polymeric material, or other types of materials. In further embodiments, one or more of the struts 109 can comprise multiple components that extend along a strut axis from the inflow end 107a to the outflow end 107b.

In some embodiments, the plurality of pivot nodes can be arranged in a plurality of columns 119 of pivot nodes that are radially spaced about the valve axis 105. As shown in FIG. 1, each column 119 of pivot nodes can be aligned along a column axis 121 that is parallel to the valve axis 105. Each column 119 of pivot nodes can each comprise a first pivot node 115a and a second pivot node 115b. In the illustrated embodiment, the first pivot node 115a can be located at the inflow end 107a of the of the radially expandable frame 103 and the second pivot node 115b can be located at the outflow end 107b of the radially expandable frame 103 although further embodiments may provide the first pivot node 115a at the outflow end and the second pivot node 115b at the inflow end. In some embodiments, the column of pivot nodes may include one or more intermediate pivot nodes positioned within the column between the first and second pivot nodes although further embodiments may not include an intermediate pivot node. For example, as shown, each column 119 of pivot nodes may include two intermediate pivot nodes 115c, 115d positioned within the column 119 between the first and second pivot nodes 115a, 115b although one intermediate pivot node or more than two intermediate pivot nodes may be provided in further embodiments.

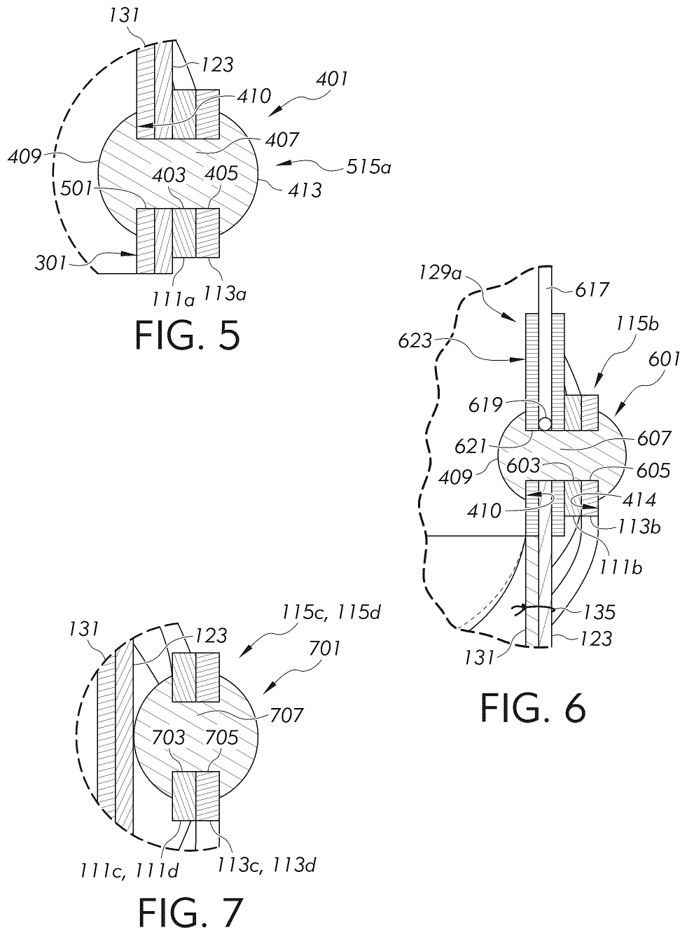

In some embodiments, the first pivot node 115a can pivotally connect a first inner strut 111a of the plurality of inner struts 111 to a first outer strut 113a of the plurality of outer struts 113 at the first pivot node 115a. As shown in FIG. 4, in some embodiments, the first pivot node 115a can comprise a first pivot pin 401 comprising a shaft 407 extending through an aperture 403 of the first inner strut 111a and an aperture 405 of the first outer strut 113a to pivotally connect the first inner strut 111a to the first outer strut 113a. FIG. 5 illustrates another embodiment of a first pivot node 515a that can also pivotally connect the first inner strut 111a to the first outer strut 113a at the first pivot node 515a. As further shown in FIG. 5, in some embodiments, the first pivot node 515a can also comprise the first pivot pin 401 comprising a shaft 407 extending through the aperture 403 of the first inner strut 111a and the aperture 405 of the first outer strut 113a to pivotally connect the first inner strut 111a to the first outer strut 113a.

In some embodiments, the second pivot node 115b can pivotally connect a second inner strut 111b of the plurality of inner struts 111 to a second outer strut 113b of the plurality of outer struts 113 at the second pivot node 115b. As shown in FIG. 6, in some embodiments, the second pivot node 115b can comprise a second pivot pin 601 comprising a shaft 607 extending through an aperture 603 of the second inner strut 111b and an aperture 605 of the second outer strut 113b to pivotally connect the second inner strut 111b to the second outer strut 113b.

In some embodiments, as shown in FIG. 7, each intermediate pivot node 115c, 115d, if provided, can pivotally connect inner struts 111c, 111d of the plurality of inner struts 111 to corresponding outer struts 113c, 113d of the plurality of outer struts 113 at each intermediate pivot node 115c, 115d. As shown, in some embodiments, the intermediate pivot nodes 115c, 115d can each comprise an intermediate pivot pin 701 comprising a shaft 707 extending through an aperture 703 of the inner struts 111c, 111d and an aperture 705 of the outer struts 113c, 113d to pivotally connect the corresponding inner struts 111c, 111d to the corresponding outer struts 113c, 113d.

The first, second, and intermediate pivot pins 401, 601, 701 can be substantially identical but may have different lengths depending on how many elements are being attached together by the pivot pin. The first pivot pin 401 pivotally attaching the first inner strut 111a to the first outer strut 113a of the first pivot node 515a will now be described with the understanding that the description can equally apply to any of the pivot pins attaching an inner strut to a corresponding outer strut of any of the pivot nodes of the disclosure. As shown in FIG. 4, the first pivot pin 401 can comprise shaft 407 with an outer dimension that can be slightly less than the dimension of the apertures 403,405 of the first inner strut 111a and the first outer strut 113a to permit relative pivoting between the shaft 407 and the first inner strut 111a and the first outer strut 113a. In some embodiments, the shaft 407 can comprise a circular cylindrical shaft and the apertures 403, 405 can comprise circular apertures. In such embodiments, the outer diameter of the circular cylindrical shaft can be slightly less than the diameter of the apertures to permit relative pivoting between the shaft 407 and the first inner strut 111a and the first outer strut 113a.

The shaft 407 of the first pivot pin 401 can be attached between an inner head 409 positioned interior of an inner surface 411a of the first inner strut 111a and an outer head 413 positioned exterior of an outer surface 415b of the first outer strut 113a. In some embodiments, the inner head 409 can comprise an outer diameter greater than the diameter of the aperture 403 of the first inner strut 111a to prevent removal of the shaft from the corresponding aligned apertures 403, 405 in an exterior direction away from the valve axis 105. Likewise, in some embodiments, the outer head 413 can comprise an outer diameter greater than the diameter of the aperture 405 of the first outer strut 113a to prevent removal of the shaft from the corresponding aligned apertures 403, 405 in an interior direction towards the valve axis 105.

The first inner strut 111a and the first outer strut 113a are trapped between the outer surface 410 of the inner head 409 and the inner surface 414 of the outer head 413. In some embodiments, the shaft 407 comprises a length between the inner surface 414 of the outer head 413 and the outer surface 410 of the inner head 409 that is large enough to permit sufficient clearance between the first inner strut 111a and first outer strut 113a to permit pivoting between the first inner strut 111a and the first outer strut 113a when radially expanding and retracting the radially expandable frame 103.

The pivot pins of the disclosure can comprise a wide range of structures to permit pivoting between the corresponding plurality of inner struts 111 and the plurality of outer struts 113. As mentioned above, the pivot pins of the disclosure can comprise the structure of the first pivot pin 401 that comprises a shaft 407 (e.g., a circular cylindrical shaft) extending between the inner and outer heads 409, 413. As shown in FIG. 4, the shaft 407 and inner and outer heads 409, 413 can be solid although the pivot pin can comprise a central passage extending through the central axis of the pivot pin in further examples. In further embodiments, the pivot pin can comprise a rivet, nut and bolt combination, or other combination. In the illustrated embodiment, the pivot pins include a single monolithic member although pivot pins can be formed from a plurality of members assembled together. In some embodiments, a single monolithic member can be crimped to provide the illustrated pivot pin pivotally connecting an inner strut 111 to a corresponding outer strut 113. In further embodiments, one or both pin heads may be provided separately and either permanently crimped, welded, screwed, press fit, otherwise connected to the shaft.

The pivot pin may be used to pivotally connect an inner strut of the plurality of inner struts 111 to a corresponding outer strut of the plurality of outer struts 113 either alone or in combination with another element. For example, as shown in FIG. 7, in some embodiments, the intermediate pin 701 of each of the intermediate pivot nodes 115c, 115d pivotally connects the corresponding third and fourth inner struts 111c, 111d to the corresponding third and fourth outer struts 113c, 113d alone without another element.

In further embodiments, as shown in FIG. 4, the first pivot node 115a can be attached to a link 123 while also pivotally attaching the first inner strut 111a to the first outer strut 113a. For example, as shown, the shaft 407 of the first pivot pin 401 of the first pivot node 115a can extend through an aperture 417 of the link 123 while also extending through the aperture 403 of the first inner strut 111a and the aperture 405 of the first outer strut 113a.

In still further embodiments, as show in FIG. 6, the second pivot node 115b can be attached to the link 123 while also pivotally attaching the second inner strut 111b to the second outer strut 113b. For example, the shaft 607 of the second pivot pin 601 of the second pivot node 115b can extend through an axial slot 617 of the link 123 to slidingly connect the second pivot node 115b to the link 123. The sliding connection of the link 123 to the second pivot node 115b is configured to facilitate movement of the second pivot node 115b relative to the link 123 along the axial direction 117.

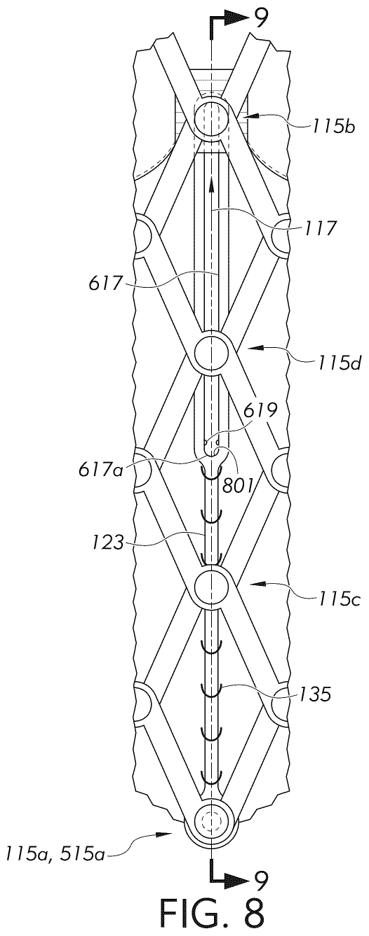

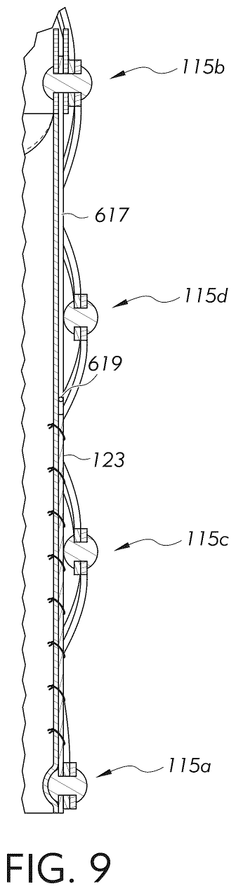

As shown in FIG. 1, the link 123 extends along the axial direction 117 and is therefore parallel with the valve axis 105. The axial slot 617 can also extend along the axial direction 117 and can comprise a closed slot with a first closed end 617a (see FIG. 8) and a second closed end 617b (see FIG. 2). The first axial closed end 617a is axially positioned between the second axial closed end 617b and the first pivot node 115a. Providing a closed slot can help trap the shaft 607 of the second pivot pin 601 of the second pivot node 115b within the slot to define a linear travel path for the second pivot node 115b in the axial direction 117.

In some embodiments, the axial slot 617 can be configured to lock a position of the second pivot pin 601 within the axial slot 617. As shown in FIG. 8, one configuration can include a pair of inward protrusions 619 defining a seat 801 although a single inward protrusion can be provided in further embodiments. A slight interference between the one or more protrusions can allow the second pivot pin 601 to pass along the axial slot 617 and into the seat 801 wherein the second pivot pin 601 is locked within the seat such that backing the second pivot pin 601 out of the seat 801 is inhibited by engagement between the shaft 607 of the second pivot pin 601 with the one or more protrusions 619. In the illustrated embodiment, the distance between the pair of protrusions 619 can be slightly less than the outer diameter of the shaft 607 of the second pivot pin 601. When the shaft 607 is within the axial slot 617 and outside of the seat 801, the second pivot pin 601 can be slide within the axial slot 617 until the shaft 607 engages the one or more protrusions 619. An additional force applied to the second pivot pin 601 can be increased until shaft 607 of the second pivot pin 601 is forced through the space provided by the one or more protrusions and into the seat 801.

Locking of the second pivot pin 601 can be maintained by the one or more protrusions 619 under a predetermined force to prevent inadvertent removal of the second pivot pin 601 from the seat 801. However, in some embodiments, a clinician may apply a force sufficient to overcome the obstruction provided by the one or more protrusions 619 such that the second pivot pin 601 is thereafter free to travel within the axial slot 617. As shown in FIG. 8, in some embodiments, the seat 801 can be provided at the first closed end 617a of the axial slot 617 such that the shaft 607 of the second pivot pin 601 can be locked within the seat as shown in FIG. 6. As the second pivot pin 601 is locked within the seat at the first closed end 617a of the axial slot 617, the prosthetic heart valve 101 can likewise be locked in the radially expanded orientation of FIG. 1. In addition or alternatively, although not shown, a similar seat can be provided at the second closed end 617b to lock the second pivot pin 601 within the second closed end to lock the prosthetic heart valve 101 in the radially retracted orientation. Although bumps are illustrated as the protrusions 619, protrusions can be provided in alternative forms such as teeth or one or more rows of protrusions designed to grip a portion of the second pivot pin. In some embodiments, the axial slot may be provided with a plurality of bumps or teeth along one or both elongated interior edges of the axial slot 617 designed to grip a portion of the second pivot pin.

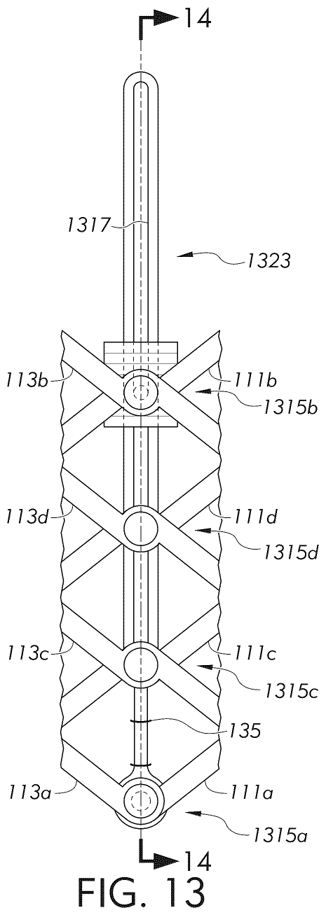

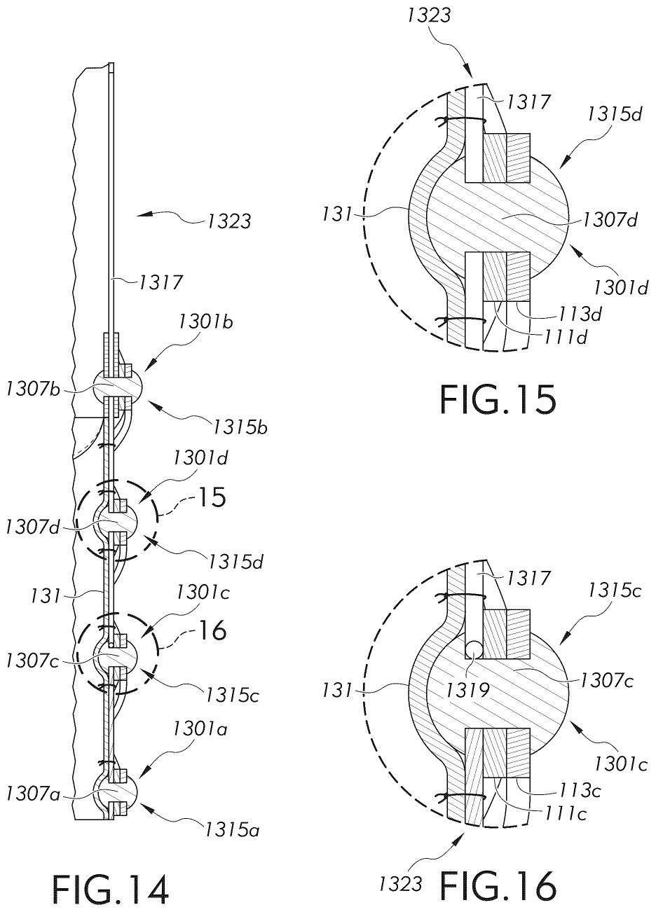

FIG. 13 illustrates another embodiment of a link 1323 that can be identical to the link 123 discussed above but includes an axial slot 1317 that is longer than the axial slot 617 of the link 123. The first pivot node 1315a comprises a first pivot pin 1301a and a shaft 1307a that is substantially similar to the first pivot node 115a, first pivot pin 401 and shaft 407 discussed above. As shown in FIGS. 14-16, the axial slot 1317 is long enough so one or more intermediate pivot nodes can be slidably connected to the link 1323 while one or more intermediate pivot node is configured to move relative to the link 1323 along the axial direction 117. In such embodiments, the one or more intermediate pivot nodes can comprise an intermediate pivot pin with a shaft extending through the axial slot to slidingly connect the intermediate pivot node to the link. For example, the axial slot 1317 can slidingly receive the shaft 1307b of the second pivot pin 1301b of the second pivot node 1315b, the shaft 1307c of the intermediate pivot pin 1301c of the third pivot node 1315c, and the shaft 1307d of the intermediate pin 1301d of the fourth pivot node 1315d. As shown in FIGS. 14-16, all of the shafts 1307c, 1307d of the corresponding pivot pins 1301c, 1301d of the corresponding third and fourth pivot nodes 1315c, 1315d are received within the axial slot 1317 although less than all of the shafts of the intermediate pivot nodes may be received within the axial slot 1317 in further embodiments. For example, the axial slot 1317 may be long enough to receive the shaft 1307d of the intermediate pivot pin 1301d of the fourth pivot node 1315d, and the shaft 1307b of the second pivot pin 1301b of the second pivot node 1315b while the shaft 1307c of the intermediate pin 1301c of the third pivot node 1315c is not received within the slot but extends outside of the slot similar to the configuration of the intermediate pivot node 115c illustrated in FIGS. 3 and 7.

As shown in FIG. 16, the axial slot 1317 can be configured to lock a position of the intermediate pin 1301c of the intermediate pivot node 115c within the axial slot 1317. In some embodiments, two protrusions 1319 (one illustrated in FIG. 16) can be provided to lock the position of the shaft 1307c of the intermediate pin 1301c within the seat. Further configurations may be provided to lock the position of the shaft 1307c in a similar or identical manner used to lock the shaft 407 of the first pivot pin 401 within the seat 801 discussed above.

The links 123, 1323 and/or the pivot pins 401, 601, 701, 1301a-d can be formed from a wide range of materials such as any of the materials used to form the plurality struts 109 such as stainless steel, nickel, cobalt, chromium, nickel titanium alloy or nitinol, polymeric material, or other types of materials. In further embodiments, the links 123, 1323 can comprise multiple components that are fastened together to provide the link. Alternatively, as shown, the links 123, 1323 can each be formed as a single component to simplify fabrication and reduce parts. Furthermore, in some embodiments, all of the links can be substantially identical regardless of whether or not the second pivot node 115b, 1315b is attached to a commissural joint as discussed more fully below.

Throughout the application, in some embodiments, the outer surface of the inner strut and the inner surface of the outer strut associated with each pivot node face one another. In further embodiments, the outer surface of the inner strut and the inner surface of the outer strut associated with each pivot node can both face and contact one another. For example, with reference to FIG. 4, in some embodiments, the outer surface 411b of the first inner strut 111a can face and abut the inner surface 415a of the first outer strut 113a. Although not shown, one or more of the pivot nodes can be provided with the outer surface of the corresponding inner strut facing the inner surface of the corresponding outer strut without contacting one another. For example, a washer (e.g., low friction washer) may be positioned between the outer surface 411b of the first inner strut 111a and the inner surface 415a of the first outer strut 113a to reduce frictional resistance to rotation that may otherwise exist. Friction reduction can also be provided between other pivoting elements of any of the pivot nodes of the disclosure. For example, with further reference to FIG. 4, a washer (e.g., low friction washer) can be provided between the link 123 and the first inner strut 111a, between the inner head 409 and the link 123 and/or between the outer head 413 and the first outer strut 113a.

In some embodiments, the prosthetic heart valves can comprise a plurality of leaflets attached at a martin of attachment to a skirt. In some embodiments, two leaflets may be provided to form a bi-leaflet (bicuspid) valve configuration. In further embodiments, three leaflets may be provided to form a tri-leaflet (tricuspid) valve configuration. Four or more leaflets may be provided in further embodiments. The skirt and/or leaflets can assume a variety of configurations, and can be formed, for example, from one or more biocompatible synthetic materials, synthetic polymers, autograft tissue, homograft tissue, xenograft tissue, or one or more other suitable materials. In some embodiments, the skirt and/or leaflets can be formed, for example, from bovine, porcine, equine, ovine and/or other suitable animal tissues. In some embodiments, the skirt and/or leaflets can be formed, for example, from heart valve tissue, pericardium, and/or other suitable tissue. In some embodiments, the skirt can comprise a fabric and can be arranged to have greater flexibility in the direction of the valve axis 105 than in a direction perpendicular to the valve axis to allow the skirt to stretch in the direction of the valve axis as the prosthetic heart valve is contracted to a contracted orientation.

As shown in FIG. 1, the prosthetic heart valve 101 is shown to include a tri-leaflet configuration although other configurations (e.g., bi-leaflet, etc.) may be provided in further embodiments. Referring to FIG. 1, the tri-leaflet configuration can comprise a first leaflet 125a, a second leaflet 125b, and a third leaflet 125c. In some embodiments, the first and second leaflets 125a, 125b can form a first pair 127a of coaptation edges, the second and third leaflets 125b, 125c can form a second pair 127b of coaptation edges, and the first and third leaflets 125a, 125d can form a third pair 127c of coaptation edges. Adjacent pairs of leaflets 125a-c are connected together by commissural joints 129a-c. In some embodiments, one or more commissural joints are connected to a second pivot node of a corresponding column 119 of commissural joints. For example, as shown in FIGS. 2, 3, and 6, a first commissural joint 129a is connected to the second pivot node 115b. As shown, in some embodiments, the shaft 607 of the second pivot pin 601 extends through an aperture 621 of the first commissural joint 129a with the inner head 409 of the second pivot pin 601 comprising an outer diameter that is greater than the diameter of the aperture 621 to prevent the pivot pin 601 from being pulled through the aperture 621 in a direction outwardly from the valve axis 105. As shown, the outer surface 410 of the inner head 409 abuts the inner surface 623 of the first commissural joint while the inner surface 414 of the outer head 413 abuts an outer surface of the second outer strut 113b. As shown in FIG. 6, the first commissural joint 129a, the link 123, the second inner strut 111b, and the second outer strut 113b are connected together by the second pivot pin 601 such that the first commissural joint 129a is configured to move together with the second pivot node 115b in an axial direction of the valve axis 105 while the shaft 607 of the second pivot pin 601 translates in the axial direction as it slides within the axial slot 617 and while the second inner strut 111b pivots relative to the second outer strut 113b about a central axis of the second pivot pin 601.

The prosthetic heart valves 101 further include a skirt 131 circumscribing an interior area defined by the radially expandable frame 103. For example, with reference to FIG. 1, the prosthetic heart valve 101 can comprise a skirt 131 that covers a portion of the inner surface of the plurality of inner struts 111. As shown in FIG. 3, an inner surface of the skirt forms an inner surface of the inner area of the prosthetic heart valve from the inflow end 107a of the radially expandable frame 103 to the leaflets 125a-c. Throughout the application, the skirt 131 can circumscribe an inner passage of the radially expandable frame 103. For example, the skirt 131 can entirely or partially circumscribes the inner passage of the radially expandable frame 103. For instance, as shown in FIG. 1, the skirt 131 is shown to circumscribe the inner passage of the radially expandable frame 103 by entirely circumscribing the inner passage of the radially expandable frame 103. Although not shown, in further embodiments, the skirt may circumscribe the inner passage of the annular from by partially (not entirely) circumscribing the inner passage of the radially expandable frame. Likewise, although the skirt 131 illustrated in FIG. 1 as extending longitudinally along the valve axis 105 entirely from the inflow end 107a to the outflow end 107b, in further embodiments, the skirt may extend partially longitudinally. For example, in some embodiments, the skirt may extend partially longitudinally from the inflow end in a direction along the valve axis and stopping short of the outflow end, partially longitudinally from the outflow end in a direction along the valve axis and stopping short of the inflow end, or partially longitudinally between at least two intermediate lateral positions located along the valve axis, each intermediate position stopping short of the inflow end and the outflow end. As further will be appreciated in FIG. 1, the skirt 131 comprises a single piece skirt 131 that circumscribes the entire interior area of the radially expandable frame 103 although the skirt 131 may comprise multiple skirts that are fastened (e.g., sewn) together to form an overall circumference that can entirely or partially circumscribe the interior area of the radially expandable frame 103.

The skirt 131 can be slidably attached to the link 123 with one or more sutures 135. As shown, a plurality of sutures 135 may be provided between the opposite ends of the link to slidably mount the skirt to the link and therefore mount the skirt indirectly to the radially expandable frame 103 by directly attaching the skirt 131 to the link. Indirectly attaching the skirt to the radially expandable frame 103 can help prevent cutting or pinching of the skirt, leaflets, or sutures that may otherwise occur due to scissor-like action between inner and outer struts.

In some embodiments, the one or more sutures 135 can comprise, for example, a flexible loop of material including but not limited to a tissue material, a fabric material, or a metal material, a synthetic or natural thread, a polymer, or other elongate material for attaching the skirt to the radially expandable frame. Moreover, although illustrated as a single suture, it should be understood that, in some embodiments, one or more of the sutures may comprise multiple individual pieces of elongate material, or a single piece of elongate material may be employed to provide one or more sutures. For example, a single loop of one piece of elongate material may define a single suture, more than one loop of one piece of elongate material may define a single suture (e.g., similar to sewing a button on a shirt), and/or a single piece of elongate material may form multiple sutures, each having one or more loops.

In some embodiments, one or more pivot nodes (e.g., pivot nodes located at the inflow end 107a) may be directly attached to the skirt although, in some embodiments, none of the pivot nodes are directly attached to the skirt. For example, as shown in FIG. 5, some embodiments may provide the inflow end of the skirt being directly attached to the first pivot node 515a (while the remaining pivot nodes in the column of pivot nodes are not attached to the skirt 131. As shown, the outer surface 410 of the inner head can abut against the inner surface 301 of the skirt 131 with the shaft 407 of the first pivot pin 401 further extending through an aperture 501 of the skirt 131. Only directly attaching the skirt 131 (i.e., the inflow end of the skirt) to the first pivot node 515a can inhibit (e.g., prevent) inverting of the inflow end of the skirt to inwardly fold the skirt back over itself such that the inflow end travels within the interior area of the radially expandable frame 103. At the same time, minimizing the number of pivot nodes being directly attached to the skirt can reduce the probability of undesired cutting or pinching of the skirt, leaflets, or sutures by the pivoting struts 109.

As further illustrated in FIG. 1, unlike the skirt 131, the leaflets 125a-c are not fixedly mounted directly to the struts 109 or the link 123. Rather, the plurality of leaflets 125a-c are each directly attached to the skirt 131 at the margin of attachment 133 without being fixedly attached to the plurality of struts 109 or the link 123. The sutures at the margin of attachment 133 can comprise a running stitch, a back stitch, or a stem stitch although the sutures of the disclosure can comprise other stitch patterns in further embodiments.

The plurality of leaflets are each tethered to the plurality of struts 109 by the skirt 131 that, in some embodiments, is slidingly attached to the link 123 by way of the sutures 135 without the margin of attachment 133 or any other part of the leaflets 125a-c being fixedly attached to the plurality of struts 109. Providing leaflets 125a-c that are indirectly attached to the struts 109 by way of tethering the leaflets 125a-c to the plurality of struts 109 with the skirt 131 and link 123 can facilitate folding and/or unfolding of the leaflets 125a-c as the radially expandable frame 103 is radially collapsed and/or radially dilated.

The radially expandable frame 103 of the prosthetic heart valves can be radially dilated and/or radially collapsed with a wide range of devices. In just one example, as shown in FIG. 1, the radially expandable frame 103 may be provided with one or more actuators. As shown, three actuators 137 can be provided although any number of actuators can be provided in further embodiments. In the illustrated embodiment, each actuator 137 can be provided with a retaining sleeve 139 attached to a pivot node (e.g., the second pivot node of a column of pivot nodes) and rotatably retaining a rod 141. The lower portion of the rod 141 can comprise exterior threads (not shown) configured to threadedly engage interior threads (not shown) of a follower 143 attached to another pivot node (e.g., the first pivot node of the column of pivot nodes). To radially expand or contract the radially expandable frame 103, a coupling 145 at the end of the rod 141 can be rotated manually or with a motor (not shown). Due to the threaded connection between the threaded portion of the rod 141 and the follower 143, rotation of the coupling 145 results in rotation of the rod 141 such that the follower 143 is threadedly driven by the rod 141 to move toward or away from the retaining sleeve 139 to radially dilate or contract the radially expandable frame 103. In addition or alternatively, in other embodiments (not shown), a mechanical tool or inflation device (e.g., balloon) configured to pivot the plurality of inner struts 111 and the plurality of outer struts 113 relative to each other at the pivot nodes can be employed to selectively radially dilate and/or contract the radially expandable frame 103.

Methods of radially expanding the prosthetic heart valve 101 will now be discussed. The method can comprise radially expanding the radially expandable frame 103 from a radially retracted orientation to a radially expanded orientation while the second pivot node 115b translates relative to the link in the axial direction 117 while being slidably connected to the link 123. The radially expandable frame 103 can be deployed from a radially retracted orientation (e.g., see FIG. 8) to a radially expanded orientation (e.g., see FIGS. 1-2) while the shaft 607 of the second pivot pin 601 slides within the axial slot 617 such that the second pivot node 115b axially translates relative to the link 123 in the axial direction 117. When radially expanding, the first inner strut 111a of the plurality of inner struts 111 can pivot relative to the first outer strut 113a of the plurality of outer struts 113 at the first pivot node 515a, wherein the first inner strut 111a and the first outer strut 113a pivot in opposite directions about a first pivot axis of the first pivot node 515a. While radially expanding, the second inner strut 111b of the plurality of inner struts 111 also pivot relative to the second outer strut 113b of the plurality of outer struts 113 at the second pivot node 513b, wherein the second inner strut 111b and the second outer strut pivot 113b pivot in opposite directions about a second pivot axis of the second pivot node 515b while the second pivot node translates relative to the link 123 in the axial direction 117 while being slidably connected to the link 123. Radial expansion can continue until the radially expandable frame 103 expands from the radially retracted orientation to the radially expanded orientation. In some embodiments, the radially expanding the radially expandable frame 103 locks the position of the shaft 607 of the second pivot pin 601 within the axial slot 617. For example, in some embodiments, once the shaft 607 reaches the inward protrusions 619, additional force can be added to force the shaft 607 through the reduced space between the inward protrusions 619 to be seated within the seat 801. Once positioned within the seat 801 of the axial slot 617, the second pivot pin 601 is locked within the axial slot 617.

In some embodiments, radially expanding the radially expandable frame axially translates the second pivot node together with the associated commissural joint relative to the link. For instance, as shown in FIG. 6, the first commissural joint 129a is attached to the second pivot node 515b with the second pivot pin 601 so that radially expanding the radially expandable frame axially translates second pivot node 515b together with the first commissural joint 129a relative to the link 123.

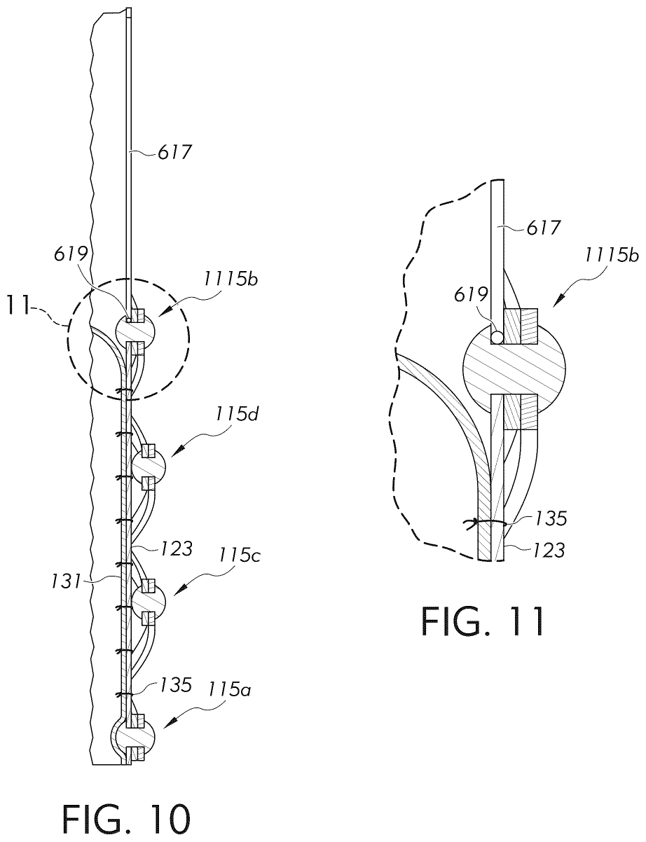

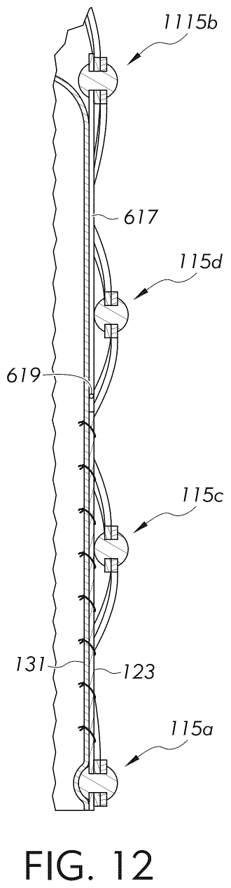

As mentioned previously and illustrated in FIG. 1, some second pivot nodes may not be connected to a commissural joint while other second pivot nodes are attached to a commissural joint. For instance, FIG. 1 illustrates three second pivot nodes that are each connected to a corresponding commissural joint of the three commissural joints and another three second pivot nodes that are not connected to a commissural joint. For example, FIGS. 3 and 6 illustrate the first commissural joint 129a connected to the second pivot node 115b while FIGS. 10-11 illustrate another embodiment of the second pivot node 1115b that is not connected to a commissural joint. Regardless of whether a commissural joint is attached to the second pivot node, radially expanding the radially expandable frame 103 can translate an attachment location of the skirt 131 relative to the link 123. For instance, as mentioned previously, the attachment location of the skirt can be movably mounted to the link 123 by the plurality of sutures 135. In such embodiments, one or more of the sutures 135 at one or more attachment locations of the skirt 131 to the link 123 can slide relative to the link 123 in the axial direction 117 when radially expanding the radially expandable frame. Movably mounting the attachment location(s) of the skirt 131 relative to the link 123 can help adjust the positioning of the skirt as the skirt is deployed with the radially expandable frame 103 expands.

In accordance with the disclosure, non-limiting aspects of the disclosure will now be described. Various combinations of the aspects can be provided in accordance with the disclosure.

Aspect 1. A prosthetic heart valve comprises a link extending along an axial direction and an axial frame. The axial frame comprises a plurality of struts comprising a plurality of inner struts pivotally attached to a plurality of outer struts at a plurality of pivot nodes. The axial frame further comprises a first pivot node of the plurality of pivot nodes pivotally connecting a first inner strut of the plurality of inner struts to a first outer strut of the plurality of outer struts at the first pivot node. The first pivot node is attached to the link. The axial frame further comprises a second pivot node of the plurality of pivot nodes pivotally connecting a second inner strut of the plurality of inner struts to a second outer strut of the plurality of outer struts at the second pivot node. The second pivot node is slidably connected to the link, wherein the second pivot node is configured to move relative to the link along the axial direction.

Aspect 2. The prosthetic heart valve of aspect 1, wherein the first pivot node comprises a first pivot pin extending through an aperture of the first inner strut and an aperture of the first outer strut to pivotally connect the first inner strut to the first outer strut, and the second pivot node comprises a second pivot pin extending through an aperture of the second inner strut and an aperture of the second outer strut to pivotally connect the second outer strut to the second inner strut.

Aspect 3. The prosthetic heart valve of aspect 2, wherein the first pivot pin extends through an aperture in the link to attach the first pivot node to the link.

Aspect 4. The prosthetic heart valve of any one of aspects 2-3, wherein the link comprises an axial slot extending along the axial direction, and the second pivot pin extends through the axial slot to slidingly connect the second pivot node to the link.

Aspect 5. The prosthetic heart valve of aspect 4, wherein the axial slot comprises a closed slot with a first axial closed end and a second axial closed end, wherein the first axial closed end is axially positioned between the second axial closed end and the first pivot node.

Aspect 6. The prosthetic heart valve of any one of aspects 4-5, wherein the axial slot is configured to lock a position of the second pivot pin within the axial slot.

Aspect 7. The prosthetic heart valve of any one of aspects 4-6, wherein the axial slot comprises at least one protrusion defining a seat to lock the second pivot pin within the seat.

Aspect 8. The prosthetic heart valve of any one of aspects 1-7, wherein the plurality of pivot nodes further comprises an intermediate pivot node positioned between the first pivot node and the second pivot node to pivotally connect a third outer strut of the plurality of outer struts to a third inner strut plurality of inner struts at the intermediate pivot node.

Aspect 9. The prosthetic heart valve of aspect 8, wherein the intermediate pivot node comprises an intermediate pivot pin extending through an aperture of the third inner strut and an aperture of the third outer strut to pivotally connect the third inner strut to the third outer strut.

Aspect 10. The prosthetic heart valve of any one of aspects 4-7, wherein the plurality of pivot nodes further comprises an intermediate pivot node positioned between the first pivot node and the second pivot node to pivotally connect a third outer strut of the plurality of outer struts to a third inner strut plurality of inner struts at the intermediate pivot node, and the intermediate pivot node is slidably connected to the link, wherein the intermediate pivot node is configured to move relative to the link along the axial direction

Aspect 11. The prosthetic heart valve of aspect 10, wherein the intermediate pivot node comprises an intermediate pivot pin extending through the axial slot to slidingly connect the intermediate pivot node to the link.

Aspect 12. The prosthetic heart valve of aspect 11, wherein the intermediate pivot pin extends through an aperture of the third inner strut and an aperture of the third outer strut to pivotally connect the third inner strut to the third outer strut.

Aspect 13. The prosthetic heart valve of any one of aspects 1-12, further comprising a plurality of leaflets, wherein two leaflets of the plurality of leaflets are connected to each other at a commissural joint that is connected to the second pivot node, wherein the commissural joint is configured to move together with the second pivot node relative to the link along the axial direction.

Aspect 14. The prosthetic heart valve of aspect 13, further comprising a skirt at least partially circumscribing an interior area defined by the radially expandable frame, the plurality of leaflets each comprise a margin of attachment to the skirt, wherein the skirt is mounted relative to the radially expandable frame.

Aspect 15. The prosthetic heart valve of aspect 14, wherein the skirt is directly attached to the link without a direct attachment to the radially expandable frame, wherein the skirt is indirectly attached the radially expandable frame by the link.

Aspect 16. The prosthetic heart valve of aspect 15, wherein the skirt is attached to the link with a plurality of sutures.

Aspect 17. The prosthetic heart valve of aspect 16, wherein at least one suture of the plurality of sutures slidably attaches the skirt to the link.

Aspect 18. The prosthetic heart valve of aspect 14, wherein the first pivot pin extends through an aperture in the skirt to directly attach the skirt to the first pivot node.

Aspect 19. A method of radially expanding the prosthetic heart valve of aspect 1, comprising radially expanding the radially expandable frame from a radially retracted orientation to a radially expanded orientation while the second pivot node translates relative to the link in the axial direction while being slidably connected to the link.

Aspect 20. A method of radially expanding the prosthetic heart valve of aspect 4, comprising radially expanding the radially expandable frame from a radially retracted orientation to a radially expanded orientation while the second pivot pin slides within the axial slot while the second pivot node axially translates relative to the link.

Aspect 21. The method of aspect 20, wherein radially expanding the radially expandable frame locks a position of the second pivot pin within the axial slot.

Aspect 22 The method of any one of aspects 19-21, wherein the prosthetic heart valve further comprises a plurality of leaflets, wherein two leaflets of the plurality of leaflets are connected to each other at a commissural joint, and radially expanding the radially expandable frame axially translates second pivot node together with the commissural joint relative to the link.

Aspect 23. The method of aspect 22, wherein the prosthetic heart valve further comprises a skirt at least partially circumscribing an interior area defined by the radially expandable frame, the plurality of leaflets each comprise a margin of attachment to the skirt, wherein an attachment location of the skirt is movably mounted to the link, wherein radially expanding the radially expandable frame translates that attachment location of the skirt relative to the link.

Aspect 24. The method of aspect 23, wherein the attachment location of the skirt is movably mounted to the link by a plurality of sutures, wherein a suture of the plurality of sutures slides relative to the link when radially expanding the radially expandable frame.

Aspect 25. A prosthetic heart valve comprises a link comprising an axial slot extending along an axial direction, and a radially expandable frame. The radially expandable frame comprises a plurality of struts comprising a plurality of inner struts pivotally attached to a plurality of outer struts at a plurality of pivot nodes. The radially expandable frame further comprises a first pivot node of the plurality of pivot nodes comprising a first pivot pin extending through an aperture of a first inner strut of the plurality of inner struts and an aperture of a first outer strut of the plurality of outer struts to pivotally connect the first inner strut to the first outer strut at the first pivot node. The first pivot node is attached to the link. The radially expandable frame further comprises a second pivot node of the plurality of pivot nodes pivotally connecting a second inner strut of the plurality of inner struts to a second outer strut of the plurality of outer struts at the second pivot node. The second pivot node comprises a second pivot pin extending through the axial slot to slidingly connect the second pivot node to the link.

The second pivot node is configured to move relative to the link along the axial direction.

Aspect 26. The prosthetic heart valve of aspect 25, wherein the second pivot pin of the second pivot node extends through an aperture of the second inner strut and an aperture of the second outer strut to pivotally connect the second outer strut to the second inner strut.

Aspect 27. The prosthetic heart valve of any one of aspects 25-26, wherein the first pivot pin extends through an aperture in the link to attach the first pivot node to the link.

Aspect 28. The prosthetic heart valve of any one of aspects 25-27, wherein the axial slot comprises a closed slot with a first axial closed end and a second axial closed end, wherein the first axial closed end is axially positioned between the second axial closed end and the first pivot node.

Aspect 29. The prosthetic heart valve of any one of aspects 25-28, wherein the axial slot is configured to lock a position of the second pivot pin within the axial slot.

Aspect 30. The prosthetic heart valve of any one of aspects 25-29, wherein the axial slot comprises at least one protrusion defining a seat to lock the second pivot pin within the seat.

Aspect 31. The prosthetic heart valve of any one of aspects 25-30, wherein the plurality of pivot nodes further comprises an intermediate pivot node positioned between the first pivot node and the second pivot node to pivotally connect a third outer strut of the plurality of outer struts to a third inner strut plurality of inner struts at the intermediate pivot node.

Aspect 32. The prosthetic heart valve of aspect 31, wherein the intermediate pivot node comprises an intermediate pivot pin extending through an aperture of the third inner strut and an aperture of the third outer strut to pivotally connect the third inner strut to the third outer strut.

Aspect 33. The prosthetic heart valve of aspect 32, wherein the intermediate pivot pin extends through the axial slot to slidably connect the intermediate pivot node to the link.

Aspect 34. The prosthetic heart valve of aspect 31, wherein the intermediate pivot node comprises an intermediate pivot pin extending through the axial slot to slidably connect the intermediate pivot node to the link.

Aspect 35. The prosthetic heart valve of any one of aspects 25-34, further comprising a plurality of leaflets, wherein two leaflets of the plurality of leaflets are connected to each other at a commissural joint that is connected to the second pivot node, wherein the commissural joint is configured to move together with the second pivot node relative to the link along the axial direction.

Aspect 36. The prosthetic heart valve of aspect 35, further comprising a skirt at least partially circumscribing an interior area defined by the radially expandable frame, the plurality of leaflets each comprise a margin of attachment to the skirt, wherein the skirt is mounted relative to the radially expandable frame.

Aspect 37. The prosthetic heart valve of aspect 36, wherein the skirt is directly attached to the link without a direct attachment to the radially expandable frame, wherein the skirt is indirectly attached the radially expandable frame by the link.

Aspect 38. The prosthetic heart valve of aspect 37, wherein the skirt is attached to the link with a plurality of sutures.

Aspect 39. The prosthetic heart valve of aspect 38, wherein at least one suture of the plurality of sutures slidably attaches the skirt to the link.

Aspect 40. The prosthetic heart valve of aspect 36, wherein the first pivot pin extends through an aperture in the skirt to directly attach the skirt to the first pivot node.

Aspect 41. A method of radially expanding the prosthetic heart valve of any one of aspects 25-40, comprising radially expanding the radially expandable frame from a radially retracted orientation to a radially expanded orientation while the second pivot node axially translates relative to the link with the second pivot pin sliding within the axial slot.

Aspect 42. The method of aspect 41, wherein radially expanding the radially expandable frame locks a position of the second pivot pin within the axial slot.

Aspect 43. A method of radially expanding the prosthetic heart valve of any one of aspects 35-40, comprising radially expanding the radially expandable frame from a radially retracted orientation to a radially expanded orientation while the second pivot node and the commissural joint axially translate together relative to the link with the second pivot pin sliding in the axial direction within the axial slot.

Aspect 44. The method of aspect 43, wherein radially expanding the radially expandable frame locks a position of the second pivot pin within the axial slot.

Aspect 45. A method of radially expanding a prosthetic heart valve comprising a radially expandable frame comprising a plurality of struts comprising a plurality of inner struts pivotally attached to a plurality of outer struts at a plurality of pivot nodes, wherein a first pivot node of the plurality of pivot nodes is connected to a link and a second pivot node of the plurality of pivot nodes is slidably connected to the link. The method comprises pivoting a first inner strut of the plurality of inner struts relative to a first outer strut of the plurality of outer struts at the first pivot node, wherein the first inner strut and the first outer strut pivot in opposite directions about a first pivot axis of the first pivot node. The method further comprises pivoting a second inner strut of the plurality of inner struts relative to a second outer strut of the plurality of outer struts at a second pivot node, wherein the second inner strut and the second outer strut pivot in opposite directions about a second pivot axis of the second pivot node while the second pivot node translates relative to the link in the axial direction while being slidably connected to the link. The radially expandable frame expands from a radially retracted orientation to a radially expanded orientation.

Aspect 46. The method of aspect 45, wherein the prosthetic heart valve further comprises a plurality of leaflets, wherein two leaflets of the plurality of leaflets are connected to each other at a commissural joint. The method further comprises radially expanding the radially expandable frame axially translates second pivot node together with the commissural joint relative to the link in the axial direction.

Aspect 47. The method of aspect 46, wherein the prosthetic heart valve further comprises a skirt at least partially circumscribing an interior area defined by the radially expandable frame, the plurality of leaflets each comprise a margin of attachment to the skirt, wherein an attachment location of the skirt is movably mounted to the link, wherein radially expanding the radially expandable frame translates that attachment location of the skirt relative to the link.

Aspect 48. The method of aspect 47, wherein the attachment location of the skirt is movably mounted to the link by a plurality of sutures, wherein a suture of the plurality of sutures slides relative to the link when radially expanding the radially expandable frame.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A prosthetic heart valve comprising:
a link extending along an axial direction; and
a radially expandable frame comprising:
a plurality of struts comprising a plurality of inner struts pivotally attached to a plurality of outer struts at a plurality of pivot nodes;
a first pivot node of the plurality of pivot nodes pivotally connecting a first inner strut of the plurality of inner struts to a first outer strut of the plurality of outer struts at the first pivot node, and the first pivot node is attached to the link; and
a second pivot node of the plurality of pivot nodes pivotally connecting a second inner strut of the plurality of inner struts to a second outer strut of the plurality of outer struts at the second pivot node, and the second pivot node is slidably connected to the link, wherein the second pivot node is configured to move relative to the link along the axial direction.

2. The prosthetic heart valve of claim 1, wherein the first pivot node comprises a first pivot pin extending through an aperture of the first inner strut and an aperture of the first outer strut to pivotally connect the first inner strut to the first outer strut, and the second pivot node comprises a second pivot pin extending through an aperture of the second inner strut and an aperture of the second outer strut to pivotally connect the second outer strut to the second inner strut.

3. The prosthetic heart valve of claim 2, wherein the first pivot pin extends through an aperture in the link to attach the first pivot node to the link.

4. The prosthetic heart valve of claim 2, wherein the link comprises an axial slot extending along the axial direction, and the second pivot pin extends through the axial slot to slidingly connect the second pivot node to the link.

5. The prosthetic heart valve of claim 4, wherein the axial slot comprises a closed slot with a first axial closed end and a second axial closed end, wherein the first axial closed end is axially positioned between the second axial closed end and the first pivot node.

6. The prosthetic heart valve of claim 4, wherein the axial slot is configured to lock a position of the second pivot pin within the axial slot.

7. The prosthetic heart valve of claim 4, wherein the axial slot comprises at least one protrusion defining a seat to lock the second pivot pin within the seat.

8. The prosthetic heart valve of claim 1, wherein the plurality of pivot nodes further comprises an intermediate pivot node positioned between the first pivot node and the second pivot node to pivotally connect a third outer strut of the plurality of outer struts to a third inner strut plurality of inner struts at the intermediate pivot node.

9. The prosthetic heart valve of claim 8, wherein the intermediate pivot node comprises an intermediate pivot pin extending through an aperture of the third inner strut and an aperture of the third outer strut to pivotally connect the third inner strut to the third outer strut.

10. The prosthetic heart valve of claim 4, wherein the plurality of pivot nodes further comprises an intermediate pivot node positioned between the first pivot node and the second pivot node to pivotally connect a third outer strut of the plurality of outer struts to a third inner strut plurality of inner struts at the intermediate pivot node, and the intermediate pivot node is slidably connected to the link, wherein the intermediate pivot node is configured to move relative to the link along the axial direction

11. The prosthetic heart valve of claim 10, wherein the intermediate pivot node comprises an intermediate pivot pin extending through the axial slot to slidingly connect the intermediate pivot node to the link.

12. The prosthetic heart valve of claim 11, wherein the intermediate pivot pin extends through an aperture of the third inner strut and an aperture of the third outer strut to pivotally connect the third inner strut to the third outer strut.

13. The prosthetic heart valve of claim 1, further comprising a plurality of leaflets, wherein two leaflets of the plurality of leaflets are connected to each other at a commissural joint that is connected to the second pivot node, wherein the commissural joint is configured to move together with the second pivot node relative to the link along the axial direction.

14. The prosthetic heart valve of claim 13, further comprising a skirt at least partially circumscribing an interior area defined by the radially expandable frame, the plurality of leaflets each comprise a margin of attachment to the skirt, wherein the skirt is mounted relative to the radially expandable frame.

15. The prosthetic heart valve of claim 14, wherein the skirt is directly attached to the link without a direct attachment to the radially expandable frame, wherein the skirt is indirectly attached the radially expandable frame by the link.

16. The prosthetic heart valve of claim 15, wherein the skirt is attached to the link with a plurality of sutures.

17. The prosthetic heart valve of claim 16, wherein at least one suture of the plurality of sutures slidably attaches the skirt to the link.

18. The prosthetic heart valve of claim 14, wherein the first pivot pin extends through an aperture in the skirt to directly attach the skirt to the first pivot node.

19. A method of radially expanding the prosthetic heart valve of claim 1, comprising radially expanding the radially expandable frame from a radially retracted orientation to a radially expanded orientation while the second pivot node translates relative to the link in the axial direction while being slidably connected to the link.

20. A method of radially expanding the prosthetic heart valve of claim 4, comprising radially expanding the radially expandable frame from a radially retracted orientation to a radially expanded orientation while the second pivot pin slides within the axial slot while the second pivot node axially translates relative to the link.
